(19) United States
(12) Patent Application Publication
(10) Pub. No.: US 2018/0296063 A1
(43) Pub. Date: Oct. 18, 2018

(54) ENDOSCOPE APPARATUS, ENDOSCOPE SYSTEM AND ENDOSCOPE IMAGE DISPLAY CONTROL METHOD

(71) Applicant: OLYMPUS CORPORATION, Tokyo (JP)

(72) Inventor: Ryoji MOTOKI, Yokohama-shi (JP)

(73) Assignee: OLYMPUS CORPORATION, Tokyo (JP)

(21) Appl. No.: 15/949,682

(22) Filed: Apr. 10, 2018

(30) Foreign Application Priority Data
Apr. 13, 2017 (JP) 2017-079570

(51) Int. Cl. A61B 1/00; H04N 5/232; A61B 1/04; A61B 1/06; G02B 23/24

(52) U.S. Cl. CPC A61B 1/0005; H04N 5/23254; H04N 5/23293; A61B 1/00006; A61B 1/00009; H04N 2005/2255; A61B 1/04; A61B 1/00016; A61B 1/0684; A61B 1/00039; G02B 23/2484; A61B 1/00096

(57) ABSTRACT

An endoscope apparatus includes an insertion portion and a body portion. The endoscope apparatus includes: an image sensor configured to pick up an image of a subject/object; and a controller configured to extract one or two or more images as one or two or more comparison images, from among a plurality of recorded images recorded in a storage apparatus, based on system configuration information indicating a configuration of the endoscope apparatus, the system configuration information being information relating to at least one of the insertion portion and the body portion, or measurement information relating to measurement processing on an endoscope image acquired in the image sensor, and perform display control to display on a display an image including the one or two or more comparison images and the endoscope image.

### CROSS REFERENCE TO RELATED APPLICATION

This application claims benefit of Japanese Application No. 2017-79570 filed in Japan on Apr. 13, 2017, the contents of which are incorporated by this reference.

### BACKGROUND

### 1. Technical Field

The present invention relates to an endoscope apparatus, an endoscope system and an endoscope image display control method.

### 2. Background Art

Conventionally, endoscope apparatuses have widely been used in the industrial field and the medical field. A user of an endoscope apparatus inserts an elongated insertion portion into an examination target and performs an endoscopic examination of the examination target while viewing an endoscope image displayed on a display.

During the endoscopic examination, the user, which is an examiner, sometimes wishes to perform observation, determination, etc., of an examined site while comparing a desired image to be compared and the endoscope image of the examination target.

For example, Japanese Patent Application Laid-Open Publication No. 2016-189812 proposes an endoscope apparatus having a comparison function that acquires an amount of insertion of an insertion portion and causes an endoscope image obtained during an examination and a past endoscope image acquired based on the amount of insertion of the insertion portion to be displayed side by side on a screen of a display.

Also, Japanese Patent Application Laid-Open Publication No. 2015-25965 proposes an endoscope apparatus having a comparison function that causes a reference image, which is a comparison image, to be displayed in such a manner that the reference image is superimposed on a live image and the live image can be seen through the reference image.

When a user wishes to compare a live image with a comparison image, the user performs an operation to execute the comparison function to cause the comparison image to be displayed on the display.

### SUMMARY

An endoscope apparatus according to an aspect of the present invention is an endoscope apparatus including an insertion portion and a body portion, the endoscope apparatus including: an image sensor configured to pick up an image of a subject/object; one or more controllers configured to: extract one or two or more images as one or two or more comparison images, from among a plurality of recorded images recorded in a storage apparatus, based on first configuration information that is information relating to at least one of the insertion portion and the body portion and is configuration information indicating a configuration of the endoscope apparatus or first measurement information that is measurement information relating to measurement processing on an endoscope image acquired in the image sensor; perform display control to display an image including the one or two or more comparison images and the endoscope image on a display.

An endoscope system according to an aspect of the present invention is an endoscope system including an endoscope apparatus including an insertion portion and a body portion, and a server connected to the endoscope apparatus via a network, wherein: the endoscope apparatus includes an image sensor configured to pick up an image of a subject/object, and one or more first controllers configured to transmit a predetermined command signal to the server; the server includes one or more second controllers configured to, upon reception of the command signal, extract one or two or more images as one or two or more comparison images, from among a plurality of recorded images recorded in a storage apparatus, based on first configuration information that is information relating to at least one of the insertion portion and the body portion and is configuration information indicating a configuration of the endoscope apparatus or first measurement information that is measurement information relating to measurement processing on an endoscope image acquired in the image sensor, and transmit the one or two or more images; and the endoscope apparatus causes an image including the one or two or more comparison images from the server and the endoscope image to be displayed on a display of the endoscope apparatus.

An endoscope apparatus according to an aspect of the present invention is an endoscope apparatus including an insertion portion and a body portion, the endoscope apparatus being connected to a server via a network, the endoscope apparatus including: an image sensor configured to pick up an image of a subject/object; and one or more controllers configured to transmit a command signal that provides an instruction to execute an extraction section configured to extract one or two or more images as one or two or more comparison images, from among a plurality of recorded images recorded in a storage apparatus, based on first configuration information that is information relating to at least one of the insertion portion and the body portion and is configuration information indicating a configuration of the endoscope apparatus or first measurement information that is measurement information relating to measurement processing on an endoscope image acquired by the image sensor, to the server including the extraction section, wherein an image including the one or two or more comparison images extracted in the server and the endoscope image is displayed on a display of the endoscope apparatus.

An endoscope image display control method according to an aspect of the present invention is an endoscope image display control method for an endoscope apparatus including an image sensor configured to pick up an image of a subject/object, an insertion portion and a body portion, the method including: extracting one or two or more images as one or two or more comparison images, from among a plurality of recorded images recorded in a storage apparatus, based on first configuration information that is information relating to at least one of the insertion portion and the body portion and is configuration information indicating a configuration of the endoscope apparatus or first measurement information that is measurement information relating to measurement processing on an endoscope image acquired in the image sensor; and displaying an image including the one or two or more comparison images and the endoscope image on a display.

### BRIEF DESCRIPTION OF THE DRAWINGS

FIG. 1 is a perspective diagram illustrating an external configuration of an endoscope apparatus according to a first embodiment of the present invention;

FIG. 2 is a block diagram illustrating an inner configuration of an endoscope apparatus 1 according to the first embodiment of the present invention;

FIG. 3 is a block diagram illustrating a configuration of a comparison image display control section 31d according to the first embodiment of the present invention;

FIG. 4 is a diagram illustrating example display of an image displayed on a display 14 upon provision of an instruction to execute a comparison function, according to the first embodiment of the present invention;

FIG. 5 is a flowchart illustrating an example flow of system configuration information acquisition processing performed when power to a body portion 3 is turned on, according to the first embodiment of the present invention;

FIG. 6 is a flowchart illustrating an example flow of endoscope image recording processing according to the first embodiment of the present invention;

FIG. 7 is a flowchart illustrating an example flow of comparison image search and display processing performed when a comparison button is selected, according to the first embodiment of the present invention;

FIG. 8 is a block diagram illustrating a configuration of a comparison image display control section 31d according to a second embodiment of the present invention;

FIG. 9 is a flowchart illustrating an example flow of comparison image search and display processing performed when a comparison button is selected, according to the second embodiment of the present invention;

FIG. 10 is a block diagram illustrating a configuration of a comparison image display control section 31d according to a third embodiment of the present invention; and

FIG. 11 is a flowchart illustrating an example flow of comparison image search and display processing performed when a comparison button is selected, according to the third embodiment of the present invention.

### DETAILED DESCRIPTION OF PREFERRED EMBODIMENTS

Embodiments of the present invention will be described below with reference to the drawings.

### First Embodiment

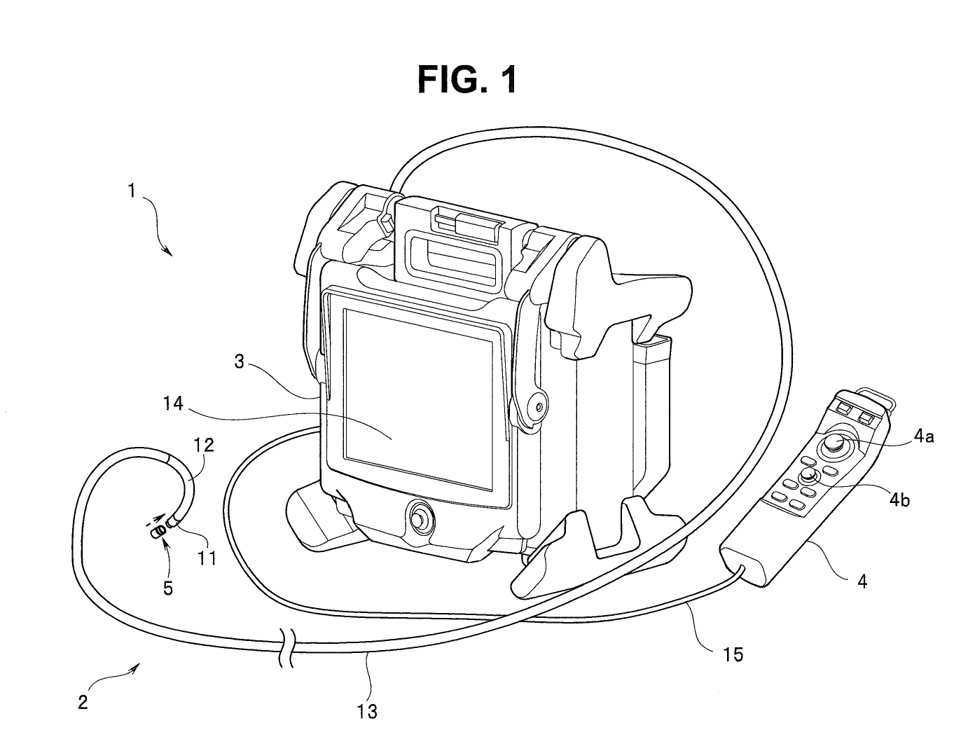

FIG. 1 is a perspective diagram illustrating an external configuration of an endoscope apparatus according to a first embodiment of the present invention.

An endoscope apparatus 1 includes an elongated insertion portion 2, a body portion 3 and an operation portion 4. The insertion portion 2 includes a distal end portion 11, a bending portion 12 and a flexible tube portion 13 continuously provided in this order from the distal end side. A proximal end of the flexible tube portion 13 is detachably connected to the body portion 3. In the distal end portion 11, as described later, an objective optical system and an image pickup device are provided.

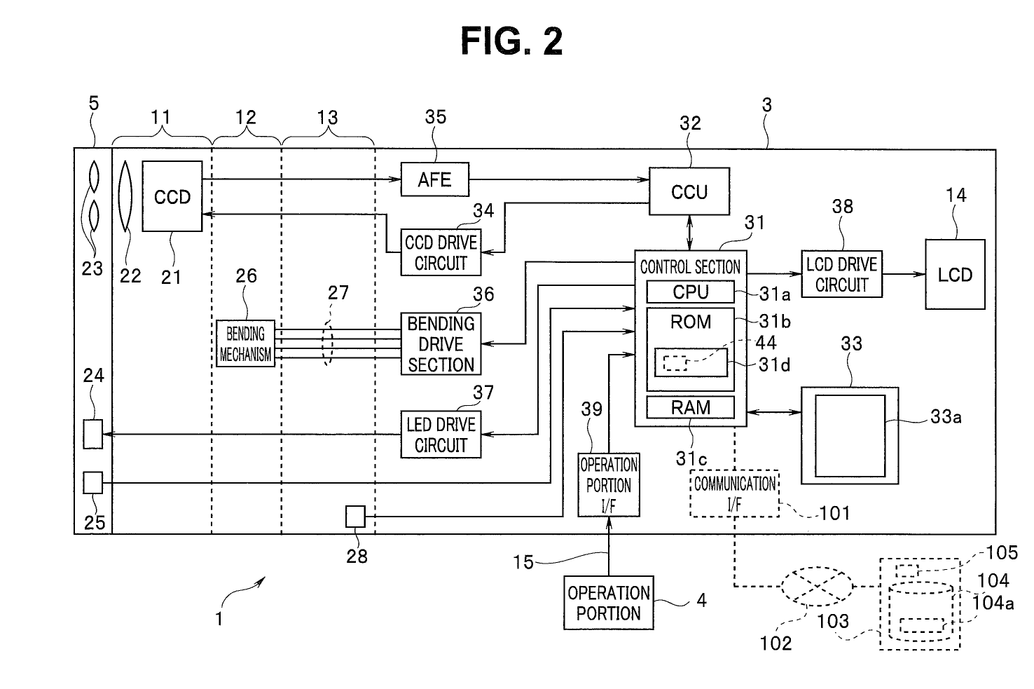

The body portion 3 includes a control section 31 (see FIG. 2) configured to control overall operation of the endoscope apparatus 1, and a liquid-crystal display (hereinafter referred to as “LCD”) 14, which is a display. An endoscope image obtained as a result of image pickup by the image pickup device of the distal end portion 11, a menu screen for a user to execute various types of functions, etc., are displayed on a screen of the LCD 14.

As indicated by the dotted arrow, an optical adapter 5 can be detachably attached to the distal end portion 11 of the insertion portion 2. As described later, the optical adapter 5 includes an optical system and a light-emitting section. A plurality of types of optical adapters 5 including, e.g., an adapter configured to change a sight direction and an adapter for measurement processing are provided. A user fits an optical adapter 5 selected according to, e.g., a purpose and/or a method of an examination to the distal end portion 11.

The operation portion 4 is connected to the body portion 3 via a cable 15. The operation portion 4 is a remote controller and includes a plurality of operation members including, e.g., various operation buttons and two joysticks 4a, 4b. The operation buttons include, e.g., a freeze button configured to display a still image, a recording button for providing an instruction to perform recording and a determination button for providing an instruction to perform determination or selection. The joystick 4a is an operation member for operating the bending portion 12 so as to bend in a desired direction, and the joystick 4b is an operation member for operation for, e.g., moving a selected item on the menu screen or moving a measurement point.

Note that it is also possible that a touch panel is attached to the LCD 14 to enable a user to operate, or provide an instruction to, the endoscope apparatus 1, together with the operation portion 4 or instead of the operation portion 4, by the user touching, e.g., a button displayed on the screen with his/her finger.

FIG. 2 is a block diagram illustrating an inner configuration of the endoscope apparatus 1.

In the distal end portion 11, a CCD image sensor (hereinafter referred to as “CCD”) 21, which serves as the image pickup device, is provided. The CCD 21 configures an image sensor configured to pick up an image of a subject/object. On the distal end side of the CCD 21, an objective optical system 22 including a plurality of lenses is disposed.

Here, the optical adapter 5 fitted to the distal end portion 11 is an adapter for measurement. The optical adapter 5 includes a lens system 23 configuring a part of the objective optical system, a light-emitting diode (hereinafter referred to as “LED”) 24, which is a semiconductor light-emitting element, and a non-volatile memory 25.

The lens system 23 includes two lenses for stereo measurement. The LED 24 is a light-emitting section for emitting illuminating light from a non-illustrated illumination window. In the memory 25, information relating to the optical adapter such as a type and/or a model of the optical adapter 5.

The information recorded in the memory 25 can be acquired by the later-described control section 31 via a signal wire provided inside the insertion portion 2 and the body portion 3 when the optical adapter 5 is fitted to the insertion portion 2 and a proximal end portion of the insertion portion 2 is connected to the body portion 3.

The bending portion 12 includes a bending mechanism 26 including a plurality of pieces. Respective distal end portions of four bending wires 27 for upward/downward/leftward/rightward bending are fixed at a most distal end piece of the bending mechanism 26. The four bending wires 27 are inserted inside the flexible tube portion 13.

A non-volatile memory 28 is provided in the proximal end portion of the insertion portion 2. In the memory 28, information relating to the insertion portion 2 such as a type and/or a model of the insertion portion 2 is recorded.

The information recorded in the memory 28 can be acquired by the later-described control section 31 via a signal wire provided inside the body portion 3 when the proximal end portion of the insertion portion 2 is connected to the body portion 3.

The body portion 3 includes the control section 31, a camera control unit (hereinafter referred to as “CCU”) 32, a memory 33, a CCD drive circuit 34, an analog front end (hereinafter referred to as “AFE”) 35, a bending drive section 36, an LED drive circuit 37, an LCD drive circuit 38 and an operation portion interface circuit (hereinafter referred to as “operation portion I/F”) 39.

The control section 31 includes, e.g., a central processing unit (hereinafter referred to as “CPU”) 31a, a ROM 31b and a RAM 31c, and performs operation control for various functions of the endoscope apparatus 1. In ROM 31b, a plurality of programs according to various functions are stored, and upon designation of a function to be executed, by a user, the CPU 31a reads a program for the function from the ROM 31b, develops the program in the RAM 31c and executes the program.

In the ROM 31b, a program for a comparison image display control section 31d is also stored. Here, the comparison image display control section 31d is a program stored in the ROM 31b, but may be configured by a circuit.

The comparison image display control section 31d provides a comparison function that causes a comparison image and a live image to be displayed side by side. The comparison function will be described later.

Here, in the ROM 31b, in addition to the program for the comparison image display control section 31d, various programs such as a display processing program for displaying an endoscope image from the CCU 32 on the LCD 14, a recording processing program for recording image data of the endoscope image from the CCU 32 in the memory 33, a reproduction processing program for reading and reproducing the image data of the endoscope image recorded in the memory 33 to display the endoscope image on the display 14 and a menu processing program for displaying the menu screen based on an instruction from the user are stored.

Therefore, the user can cause a live endoscope image or the menu screen to be displayed on the screen 14a or provide an instruction to execute the comparison function during execution of a measurement function, by operating the operation portion 4.

Furthermore, in ROM 31b, information relating to the body portion 3 such as a serial number of the body portion 3 is also stored.

The CCU 32 is a circuit configured to receive an image pickup signal from the CCD 21 and generate an endoscope image. The CCU 32 generates and outputs a drive signal for the CCD drive circuit 34 under the control of the control section 31. The CCU 32 receives an image pickup signal from the AFE 35, generates an endoscope image that is a live image and outputs the endoscope image to the control section 31 under the control of the control section 31.

The memory 33 is a rewritable non-volatile memory, for example, a memory card that incorporates a flash memory. Here, the memory 33 is attachable/detachable to/from the endoscope apparatus 1. The memory 33 includes a storage area 33a that can record, e.g., data of images. The user can record a still image or a movie of the endoscope image in the memory 33 by operating the operation portion 4.

Recording of an image in the memory 33 and reading of an image from the memory 33 are performed under the control of the control section 31.

The CCD drive circuit 34 is connected to the CCD 21 via a signal wire, generates various drive signals for driving the CCD 21 and outputs the drive signals to the CCD 21.

The AFE 35 is connected to the CCD 21 via a signal wire. The AFE 35 receives an image pickup signal, which is an analog signal from the CCD 21, subjects the image pickup signal to processing such as analog-digital conversion and denoising and outputs the resulting image pickup signal to the CCU 32.

The bending drive section 36 includes motors and a mechanism that pulls and loosens the respective bending wires 27. The bending drive section 36 pulls and loosens the respective bending wires 27 via the motors under the control of the control section 31. Upon the user operating the joystick 4a, the control section 31 receives an operation signal from the joystick 4a of the operation portion 4 and outputs a drive signal to the motors according to the operation signal.

The LED drive circuit 37, which operates under the control of the control section 31, generates a drive signal and outputs the drive signal to the LED 24. The LED 24 emits light according to the drive signal, and the light of the LED 24 is emitted from an illumination window (not illustrated) as illuminating light.

The LCD drive circuit 38 receives an image signal from the control section 31 and outputs the image signal to the LCD 14.

The operation portion I/F 39 is a circuit configured to generate operation signals representing operations of various operation buttons and the joysticks 4a, 4b of the operation portion 4 and output the operation signals to the control section 31.

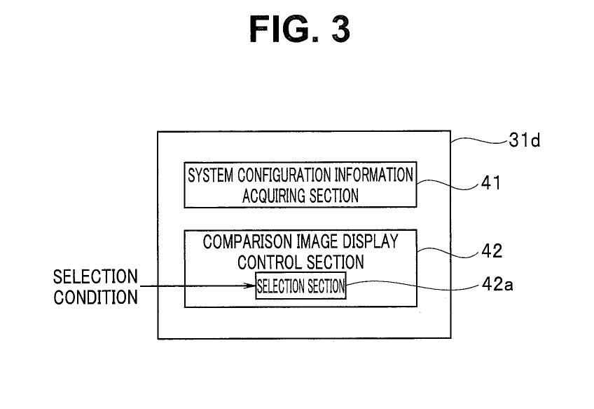

Next, a configuration of the comparison image display control section 31d that provides the comparison function of the endoscope apparatus 1 will be described. FIG. 3 is a block diagram illustrating a configuration of the comparison image display control section 31d.

The comparison image display control section 31d includes a system configuration information acquiring section 41 and a comparison image display control section 42.

System configuration information includes the information relating to each of the insertion portion 2, the body portion 3 and the optical adapter 5. The system configuration information is information indicating the configuration of the endoscope apparatus. The system configuration information acquiring section 41 is a processing program for acquiring the system configuration information.

The information relating to the optical adapter in the system configuration information is the information including a type such as a monocular adapter, a stereo adapter or a special adapter. Here, since the optical adapter 5 is a stereo adapter that is an adapter for measurement, the information relating to the optical adapter is the information indicating, e.g., the type of the stereo adapter. The information relating to the body portion 3 is the information indicating, e.g., a serial number, a grade and a version of the body portion. The information relating to the insertion portion 2 is the information relating to, e.g., a diameter and a length of the insertion portion.

The comparison image display control section 42 is a processing section configured to generate and output image data of an image including a comparison image and a live image (hereinafter referred to as “comparison function image”). In other words, a comparison function image is displayed on the screen 14a as an image in which a comparison image and a live image are arranged side by side. A content of processing in the comparison image display control section 42 will be described later.

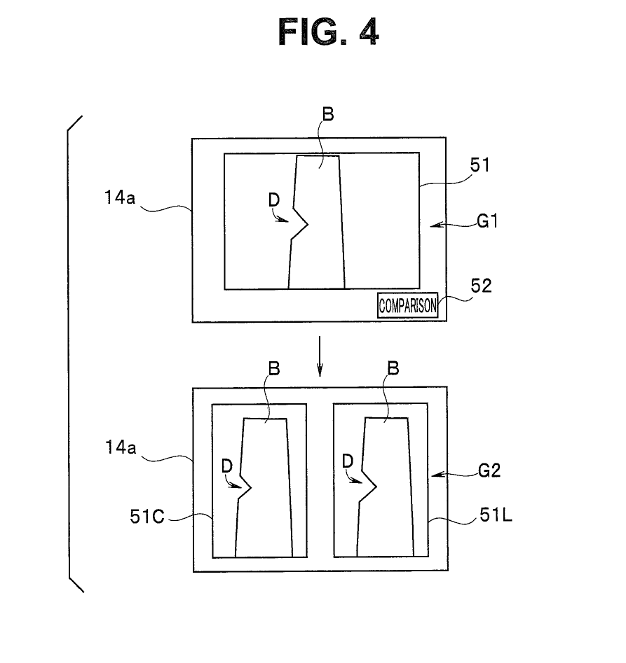

FIG. 4 is a diagram illustrating example display of an image displayed on the display 14 upon provision of an instruction to execute the comparison function.

The user can provide an instruction to execute the comparison function when the measurement function is being executed during observation of an endoscope image. FIG. 4 indicates transition of an image displayed on the screen 14a of the display 14.

An image G1 on the upper side of FIG. 4 is an image showing an endoscope image before provision of an instruction to execute the comparison function, and includes one image display area 51, and the endoscope image that is a live image is displayed in the image display area 51. Furthermore, in the image G1, a comparison button 52 for providing an instruction to execute the comparison function is displayed. In FIG. 4, an endoscope image including a blade B inside an aircraft engine that is an examination target in the image display area 51 is illustrated.

The user can provide an instruction to execute the comparison function by providing an instruction to select and execute the comparison button 52 via operations such as positioning a cursor displayed on the screen 14a over the comparison button 52 and pressing the determination button of the operation portion 4.

Upon provision of an instruction to execute the comparison function, an image G2 on the lower side of FIG. 4 is displayed on the screen 14a instead of the image G1. The image G2 includes two image display areas 51L, 51C that display two images, and an endoscope image that is a live image and a comparison image are displayed side by side in the two image display areas 51L and 51C, respectively.

In the image display area 51L, a live image obtained as a result of image pickup by the CCD 21 is displayed. As described later, the comparison image displayed in the image display area 51C is a past endoscope image selected based on, e.g., the system configuration information.

Therefore, the user can determine a state of an examination target by executing the comparison function and comparing a live image and a past recorded image.

More specifically, for example, in the case of FIG. 4, the user cannot recognize a change in size of a chip part D created in the blade B of the aircraft engine that is an examination target, by viewing the endoscope image displayed in the image display area 51 of the image G1 alone, but can determine a degree of the change in size of the chip part D through passage of time from a past examination to the present by comparing the comparison image displayed in the image display area 51C and the live image displayed in the image display area 51L, which are arranged side by side by means of the comparison function.

Note that, in the case of FIG. 4, one image display area 51C that displays a comparison image is provided, but two or more image display areas 51C may be provided.

Therefore, the comparison image display control section 42 configures a display control section configured to perform display control to display an image including one or two or more comparison images and an endoscope image on the LCD 14, which is a display.

Referring back to FIG. 3, as described later, a selection section 42a is a processing section configured to select a comparison image to be displayed in the image display area 51C from among a plurality of comparison images searched for and extracted based on the system configuration information from among a plurality of endoscope images recorded in the memory 33, according to a set selection condition. The user can set and input a selection condition in the selection section 42a. Selection condition data is stored in a storage area in the memory 33, the storage area being managed by the selection section 42a.

The user can input, for example, a designated date or either ascending order or descending order of dates to the selection section 42a as a selection condition by operating the operation portion 4.

The selection condition may be, e.g., measurement information, additional information, file name information, image information, file type information, file position information or folder designation information.

The measurement information is information relating to endoscope image measurement obtained when the measurement function was used and is information on, for example, a measurement method (e.g., measurement of a distance between two points, an object distance from the distal end portion 11 to an object and/or area measurement), measurement sites (e.g., positions and/or the number of measurement points on the image) and/or measurement results (e.g., numerical values).

The additional information is information added to an endoscope image by a user and is information such as various comments and examination result-related information. Each comment is text information arbitrarily added to the relevant image by the user. The examination result-related information is information indicating examination results (e.g., no abnormality found, a crack found or a chip found) or the like.

The file name information is information on a file name of a file of an endoscope image. The file name is information for uniquely designating the file.

The image information is information on, for example, a video format, a video size, a frame rate and/or a zoom amount of an endoscope image.

The file type information is information on a type such as a still image, a movie, an image with sound and a three-dimensional image.

The file position information is information on a position of a comparison image folder used when a plurality of comparison image folders are set, for example, information for designating either of the memory 33 of the body portion 3 and a memory in an apparatus on a network.

The folder designation information is information on a name of a comparison image folder used when a plurality of comparison image folders are set, such as a folder storing an image in a normal state or a folder storing an image of a last examination result.

When a comparison image is selected according to the system configuration information and the selection condition, the comparison image does not always exactly match the system configuration information and the selection condition, but may partly match the system configuration information and the selection condition, and in such a case, one or two or more comparison images are selected in descending order of degree of coincidence.

For example, a selection order of the found one or two or more comparison images is determined according to a descending order of the number of coincident system configuration information pieces so that a comparison image having a larger number of coincident system configuration information pieces is given a higher rank and a comparison image having a smaller number of coincident system configuration information pieces is given a lower rank.

It is also possible that information types (information relating to the insertion portion 2, the body portion 3 and the optical adapter 5) in the system configuration information are prioritized and a selection order is determined in descending order of degree of coincidence of system configuration information of a type having a higher priority.

### (Operation)

Next, comparison function image display control processing will be described. First, system configuration information acquisition processing when power is on will be described.

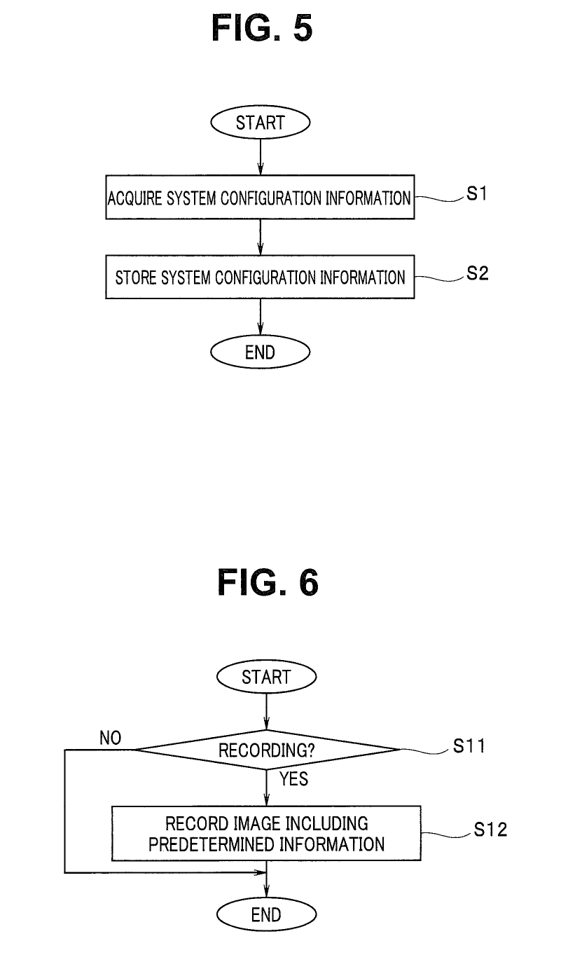

FIG. 5 is a flowchart illustrating an example flow of system configuration information acquisition processing executed when power to the body portion 3 is turned on. The processing in FIG. 5 is performed by the system configuration information acquiring section 41.

The processing in FIG. 5 is performed when the user connects the flexible tube portion 13 of the endoscope to the body portion 3 and fits the optical adapter 5 to the distal end portion 11 of the insertion portion 2 and then turns on the power to the body portion 3.

The system configuration information acquiring section 41 acquires the information relating to the optical adapter 5 from the memory 25, the information relating to the insertion portion 2 from the memory 28 and the information relating to the body portion 3 from a predetermined address in the ROM 31b, as system configuration information (step (hereinafter abbreviated as “S”) 1).

The system configuration information acquiring section 41 stores the information relating to the optical adapter 5, the information relating to the insertion portion 2 and the information relating to the body portion 3 acquired in S1, in the RAM 31c inside the control section 31, as system configuration information (S2).

As a result of the processing in FIG. 5, the system configuration information is recorded in the RAM 31c, which is a storage section.

Note that the system configuration information acquisition processing is performed when the power to the body portion 3 is on, but may be performed after a lapse of a predetermined time period from when the power is turned on or, as described later, may be performed when the comparison button 52 is selected.

Next, a flow of processing when the user records an endoscope image of an examination target will be described.

FIG. 6 is a flowchart illustrating an example flow of endoscope image recording processing. FIG. 6 indicates processing performed by a processing program stored in the ROM 31b, and the processing is constantly performed during an examination.

The control section 31 determines whether or not an instruction to perform recording is given (S11). More specifically, in order to determine whether or not the user has provided an instruction to perform image recording, whether or not the user has operated a predetermined button, for example, the recording button, of the operation portion 4 is determined.

If no instruction to perform recording has been provided (S11: NO), the control section 31 performs no particular processing. If an instruction to perform recording has been provided (S11: YES), the control section 31 records an image including predetermined information (S12).

In general, an endoscope image is recorded in the memory 33 in the form of image data of a format such as JPEG or TIFF. Image file formats include a format in which image data can be recorded with information relating to the relevant image, for example, EXIF information, added, and, for example, image data of, e.g., JPEG and TIFF have such format.

If the user provides an instruction to perform image recording, the control section 31 reads the system configuration information stored in the RAM 31c and records image data including predetermined information including the system configuration information as EXIF information, in the memory 33 (S12). The recorded image is a still image or a movie. Upon provision of an instruction to perform image recording when the user is using the measurement function, the control section 31 records image data including predetermined information including the system configuration information and measurement information as EXIF information, in the memory 33 (S12). The measurement information is information on, e.g., a measurement method, a measurement site and/or a measurement result, which has been described above.

In S12, as the predetermined information, in addition to the system configuration information, e.g., the measurement information, additional information, file name information, image information, file type information, file position information and/or folder designation information, which have been described above, are recorded in the memory 33.

Therefore, the processing in S12 configures an image recording section configured to add system configuration information when an endoscope image is acquired, to the acquired endoscope image and record the endoscope image in the memory 33.

Next, processing for comparison image search and display based on system configuration information will be described.

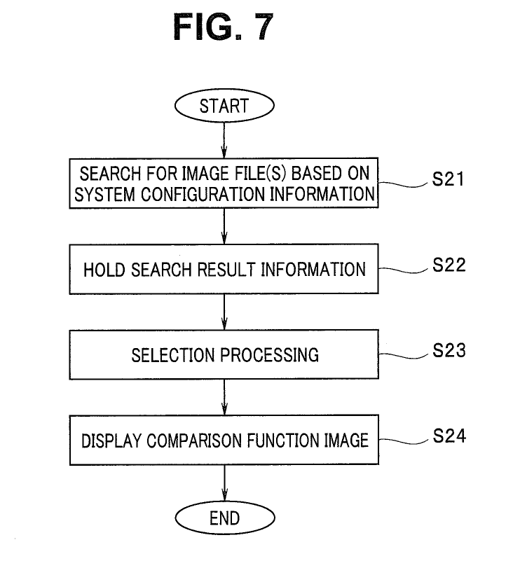

FIG. 7 is a flowchart illustrating an example flow of comparison image search and display processing performed when the comparison button is selected. The processing in FIG. 7 is performed by the comparison image display control section 42.

As described above, upon the user providing an instruction to select and execute the comparison button 52 to provide an instruction to execute the comparison function, the comparison image display control section 42 reads the system configuration information stored in the RAM 31c and searches for an image file based on the system configuration information (S21).

As described above, the image file includes image data that is data of the image itself and information added to the image data as, for example, EXIF information.

As described above, the system configuration information is the information relating to the optical adapter 5, the insertion portion 2 and the body portion 3. Therefore, the comparison image display control section 42 extracts an image file including system configuration information that matches or is similar to the system configuration information from among image data of one or two or more past endoscope images recorded in the memory 33.

Therefore, the comparison image display control section 42 configures an extraction section configured to extract one or two or more images as one or two or more comparison images, from among a plurality of recorded images recorded in the memory 33, which is a storage apparatus, based on configuration information indicating the configuration of the endoscope apparatus 1, the configuration information being the information relating to the insertion portion 2, the body portion 3 and the optical adapter 5.

In this case, the comparison image display control section 42 reads system configuration information in EXIF information included in image data of each past endoscope image recorded in the memory 33, calculates a degree of coincidence between the system configuration information and the system configuration information on the endoscope apparatus 1 stored in the RAM 31c and extracts each image file having a degree of coincidence that is equal to or exceeds a predetermined threshold value TH1. In other words, the comparison image display control section 42 extracts one or two or more comparison images based on a degree of coincidence between the configuration information stored in the RAM 31c and the configuration information added to each recorded image.

Next, the comparison image display control section 42 holds search result information in the RAM 31c (S22). The information relating to the image data extracted according to the degree of coincidence of system configuration information is stored in the RAM 31c.

The search result information may be file data of the one or two or more endoscope images searched for and extracted from among the plurality of endoscope images stored in the memory 33 or may be file address information of the one or two or more endoscope images. For example, in S22, file data of the plurality of endoscope images are stored in the RAM 31c in such a manner that the file data are arranged in descending order from file data having perfectly matched system configuration information or descending order from file data having system configuration information not perfectly matching the system configuration information but having a higher degree of coincidence with the system configuration information.

The comparison image display control section 42 performs selection processing (S23). In the selection processing, one comparison image is selected and read from the data file of the one or two or more images extracted in S12, according to a selection condition set in the selection section 42a.

After S23, the comparison image display control section 42 generates image data for the image G2 in FIG. 4 using the read comparison image and outputs the image data to the LCD drive circuit 38 to display a comparison function image (S24). More specifically, the comparison image display control section 42 generates the image G2 in FIG. 4 based on the comparison image selected and read in S23 and a live image from the CCU, and outputs the image data of the image G2 to the LCD drive circuit 38. As a result, the image G2 in FIG. 4 is displayed on the screen 14a of the LCD 14.

Note that, if only one comparison image is selected in the selection section 42a, the comparison image alone is displayed in the image display area 51C. If a plurality of comparison images are selected in the selection section 42a, one comparison image selected according to the degree of coincidence, from among the comparison images selected according to the selection condition, is displayed in the image display area 51C. In such a case, the user can cause a comparison image determined as having a highest degree of coincidence next to the currently displayed comparison image or a comparison image determined as having a lowest degree of coincidence next to the currently displayed comparison image to be displayed in the image display area 51C, by operating a predetermined operation member, for example, the joystick 4b, of the operation portion 4.

Note that, where two or more image display areas 51C that each display a comparison image are provided, two or more comparison images each having a high degree of coincidence are displayed in the plurality of image display areas 51C.

Note that the processing in S21 and S22 and the processing in S21 to S23 may be performed in advance to selection of the comparison button, for example, when the power is turned on.

As described above, the above embodiment enables provision of an endoscope apparatus and an endoscope image display control method enabling a comparison image to be displayed easily.

Upon execution of the comparison function during an examination by the user, a comparison image is automatically searched for and displayed based on the system configuration information of the endoscope apparatus 1.

Then, if a plurality of comparison images are found based on system configuration information, a comparison image selected according to a predetermined selection condition is displayed in the image display area 51C of the image G2. When a plurality of comparison images are extracted, if the user wishes to cause another comparison image not displayed in the image display area 51C to be displayed in the image display area 51C of the image G2, the user can cause a next comparison image to be displayed in the image display area 51C, by operating the joystick 4b of the operation portion 4.

Therefore, upon execution of the comparison function by the user, a related past comparison image is displayed in the image G2, which is a comparison function image, eliminating the need for complicated operation.

Note that although in the above-described example, information relating to three components, that is, the insertion portion 2, the body portion 3 and the optical adapter 5, is used as configuration information relating to the configuration of the endoscope apparatus and one or two or more comparison images are extracted based on the degree of coincidence of information relating to the three components, it is possible that only information relating to two components of the three components is used and one or two or more comparison images are extracted based on the degree of coincidence with the information relating to the two components.

Furthermore, it is possible that only information relating to one component of the three components is used and one or two or more comparison images are extracted based on the degree of coincidence of the information relating to the one component.

### Second Embodiment

Although in the first embodiment, a comparison image is searched for and displayed based on system configuration information, in the present embodiment, a comparison image is searched for and displayed based on measurement information.

As an endoscope system according to the present embodiment has a configuration that is similar to the configuration of the endoscope system according to the first embodiment, components that are the same as components of the first embodiment are provided with reference numerals that are the same as reference numerals of the first embodiment and description of such components will be omitted and only components that are different from components of the first embodiment will be described.

The endoscope system according to the present embodiment has a configuration that is the same as the configuration illustrated in FIGS. 1 and 2, but is different from the configuration illustrated in FIGS. 1 and 2 in configuration of a comparison image display control section 31d.

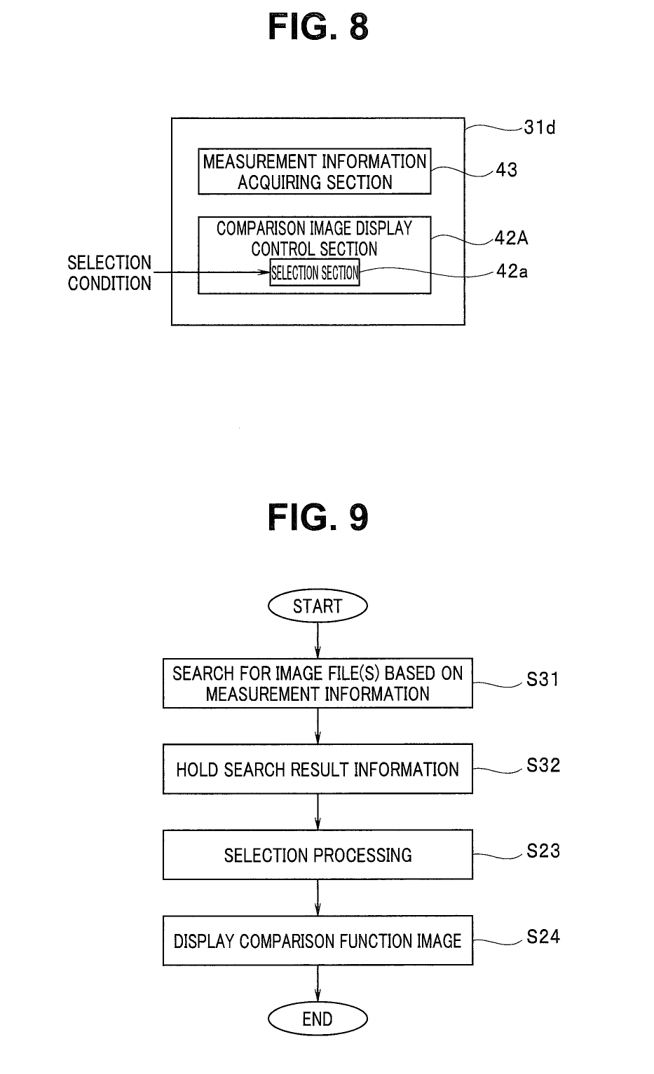

FIG. 8 is a block diagram illustrating a configuration of a comparison image display control section 31d according to the present embodiment.

The comparison image display control section 31d includes a measurement information acquiring section 43 and a comparison image display control section 42A.

During execution of a measurement function, a user designates, e.g., a measurement method and a measurement site and performs measurement. The measurement information acquiring section 43 acquires, e.g., information on the measurement method and the measurement site designated by the user in the measurement function and measurement result information, such as a distance and/or a length, obtained as a result of the measurement.

As with the comparison image display control section 42, the comparison image display control section 42A is a processing section configured to generate and output image data of a comparison function image. In other words, a comparison function image is displayed on a screen 14a as an image in which a comparison image and a live image are arranged side by side. A content of the processing in the comparison image display control section 42A will be described later.

In the endoscope system according to the present embodiment, also, an endoscope image is acquired in such a manner as described with reference to FIG. 6 in the first embodiment and is recorded in a memory 33. Therefore, measurement information is added to image data of the endoscope image and the image data is recorded in the memory 33.

Next, comparison image search and display processing on recorded endoscope images based on measurement information will be described.

FIG. 9 is a flowchart illustrating an example flow of comparison image search and display processing performed when a comparison button is selected. The processing in FIG. 9 is performed by the comparison image display control section 42A.

In FIG. 9, processing in a step that is the same as processing in a step in FIG. 6 is provided with a step number that is the same as the number of the step in FIG. 6.

Upon the user selecting a comparison button 52 to provide an instruction to execute a comparison function as described above, the comparison image display control section 42A searches for an image file based on measurement information including, e.g., information such as a measurement method and/or a measurement site designated in the measurement function and measurement result information such as a distance and/or a length obtained as a result of measurement (S31).

For the image file searching, an image file including measurement information that matches or is similar to the measurement information relating to the measurement function used by the user is extracted from among image data recorded in a storage area 33a of the memory 33.

As described above, when an endoscope image is recorded, predetermined information including measurement information is also recorded in the relevant image data as, for example, EXIF information. Therefore, in S31, the comparison image display control section 42A reads measurement information in EXIF information included in image data of each past endoscope image recorded in the memory 33, calculates a degree of coincidence between the measurement information and measurement information including information such as a measurement method for an image displayed on the screen 14a and extracts each image file having a degree of coincidence that is equal to or exceeds a predetermined threshold value TH2.

Therefore, the comparison image display control section 42A configures an extraction section configured to extract one or two or more images as one or two or more comparison images, from among a plurality of recorded images recorded in the memory 33, which is a storage apparatus, based on measurement information relating to measurement processing for an endoscope image acquired in a CCD 21.

In this case, the comparison image display control section 42A reads measurement information in EXIF information included in image data of each past endoscope image recorded in the memory 33, calculates a degree of coincidence between the measurement information and measurement information relating to measurement processing for an endoscope image acquired in the CCD 21 and extracts each image file having a degree of coincidence that is equal to or exceeds the predetermined threshold value TH2. In other words, the comparison image display control section 42A extracts one or two or more comparison images based on a degree of coincidence between the measurement information relating to measurement processing for an endoscope image acquired in the CCD 21 and the measurement information added to each recorded image.

Next, the comparison image display control section 42A holds a search result information in a RAM 31c (S32). Information relating to image data extracted according to the degree of coincidence of the measurement information is stored in the RAM 31c.

The search result information may be file data of one or two or more endoscope image searched for and extracted from among the plurality of endoscope images stored in the memory 33 or may be file address information of the one or two or more endoscope images. For example, in S32, file data of the plurality of endoscope images are stored in the RAM 31c in such a manner that the file data are arranged in descending order from file data having perfectly matched measurement information or descending order from file data having measurement information not perfectly matching the measurement information but having a higher degree of coincidence with the measurement information.

The comparison image display control section 42A performs selection processing (S23). In the selection processing, one comparison image is selected and read from the file data of the one or two or more images extracted in S32, according to selection condition set in a selection section 42a.

After S23, the comparison image display control section 42A generates image data for the image G2 in FIG. 4 using the read comparison image and outputs the image data to an LCD drive circuit 38 to display a comparison function image (S24). More specifically, the comparison image display control section 42A generates the image G2 in FIG. 4 based on the comparison image selected and read in S23 and a live image from a CCU and outputs image data of the image G2 to the LCD drive circuit 38. As a result, the image G2 in FIG. 4 is displayed on the screen 14a of the LCD 14.

Note that, as in the first embodiment, in the present embodiment, also, if only one comparison image is selected in the selection section 42a, the comparison image alone is displayed in an image display area 51C. When a plurality of comparison images are selected in the selection section 42a, one comparison image selected according to the degree of coincidence, from among the comparison images selected according to the selection condition, is displayed in the image display area 51C. In such a case, the user can cause a comparison image determined as having a highest degree of coincidence next to the currently displayed comparison image or a comparison image determined as having a lowest degree of coincidence next to the currently displayed comparison image to be displayed in the image display area 51C, by operating a predetermined operation member, for example, a joystick 4b of an operation portion 4.

Note that when two or more image display areas 51C that each display a comparison image are provided, two or more comparison images each having a high degree of coincidence are displayed in the plurality of image display areas 51C.

As described above, the above embodiment enables provision of an endoscope apparatus and an endoscope image display control method enabling a comparison image to be displayed easily.

Upon the user executing the comparison function during use of the measurement function in an examination, a comparison image is automatically searched for and displayed based on the measurement information of the endoscope apparatus 1.

Then, if a plurality of comparison images are searched for based on the measurement information, a comparison image selected according to a predetermined selection condition is displayed in the image display area 51C of the image G2. When a plurality of comparison images are extracted, if the user wishes to cause another comparison image not displayed in the image display area 51C to be displayed in the image display area 51C of the image G2, the user can cause a next comparison image to be displayed in the image display area 51C, by operating the joystick 4b of the operation portion 4.

Therefore, upon execution of the comparison function by the user, a related past comparison image is displayed in the image G2, which is a comparison function image, eliminating the need for complicated operation.

Note that although in the above-described example, one or two or more comparison images are extracted based on the degree of coincidence of information on, e.g., the measurement method and the measurement site designated in the measurement function and/or measurement results such as the distance and/or the length obtained as a result of measurement, as measurement information, one or two or more comparison images may be extracted based on the degree of coincidence of information relating to at least one of the measurement method and the measurement site designated in the measurement processing and the measurement results.

### Third Embodiment

Although in the first embodiment, a comparison image is searched for based on system configuration information and displayed and in the second embodiment, a comparison image is searched for based on measurement information and displayed, in the present embodiment, after past endoscope images are searched for based on system configuration information, a comparison image is further searched for based on measurement information and displayed.

An endoscope system according to the present embodiment has a configuration that is similar to the configurations of the endoscope systems according to the first and second embodiments. Thus components that are the same as components in the first and second embodiments are provided with reference numerals that are the same as reference numerals of the components in the first and second embodiments, description of such components will be omitted and only components that are different from components in the first and second embodiments will be described.

The endoscope system according to the present embodiment has a configuration that is the same as the configuration illustrated in FIGS. 1 and 2, but is different from the configuration illustrated in FIGS. 1 and 2 in configuration of a comparison image display control section 31d.

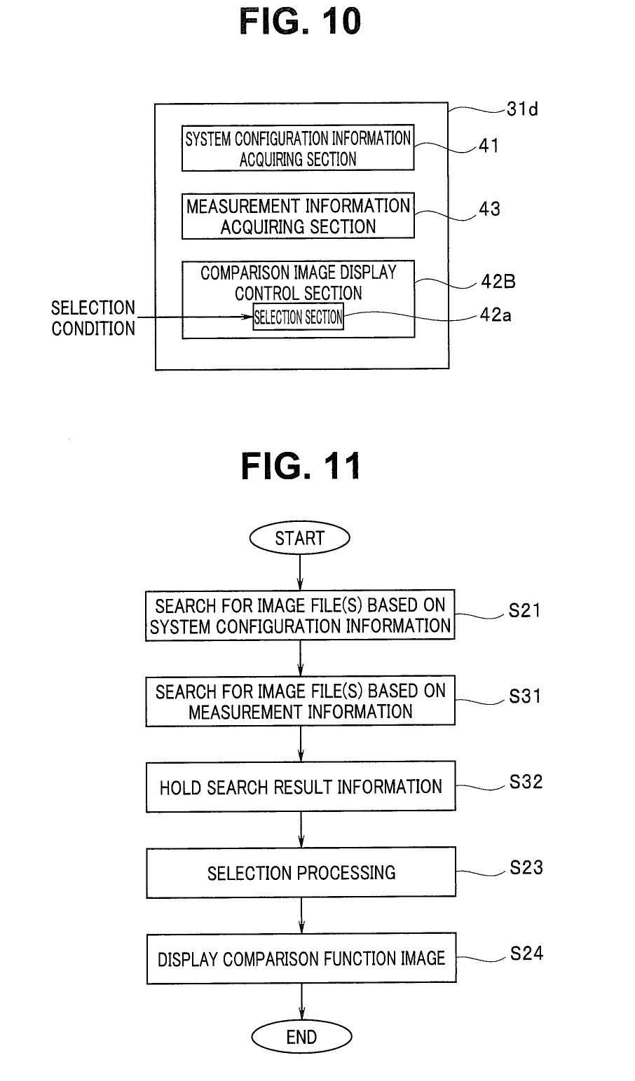

FIG. 10 is a block diagram illustrating a configuration of a comparison image display control section 31d according to the present embodiment.

The comparison image display control section 31d includes a system configuration information acquiring section 41, a measurement information acquiring section 43 and a comparison image display control section 42B.

As with the comparison image display control sections 42, 42A, the comparison image display control section 42B is a processing section configured to generate and output image data of a comparison function image. In other words, a comparison function image is displayed on a screen 14a as an image in which a comparison image and a live image are arranged side by side.

As illustrated in FIG. 5, system configuration information is acquired, for example, when power to the body portion 3 is turned on.

As illustrated in FIG. 6, image recording is performed by, upon provision of an instruction to record an image, recording image data including predetermined information such as system configuration information and measurement information in a memory 33.

FIG. 11 is a flowchart illustrating an example flow of comparison image search and display processing executed when a comparison button is selected. The processing in FIG. 11 is performed by the comparison image display control section 42B.

In FIG. 11, processing that is the same as processing in a step in FIGS. 6 and 9 is provided with a step number that is the same as the step number of the step in FIGS. 6 and 9.

As illustrated in FIG. 11, first, the comparison image display control section 42B performs the processing in S21 to search for and extract an image file acquired in a system that is the same as or similar to, in system configuration, the endoscope system according to the present embodiment, from among image data in the memory 33.

Next, the image data extracted in S21 is subjected to the processing in S31 to search for file data of each image file including measurement information that matches or is similar to measurement information relating to a currently used measurement function.

In other words, the comparison image display control section 42B configures an extraction section configured to extract one or two or more comparison images, from among a plurality of recorded images, based on configuration information and subsequently extract one or two or more comparison images, from among the extracted one or two or more comparison images, based on measurement information.

Subsequently, the comparison image display control section 42B performs processing in S32, S23 and S24.

As a result, the image G2 in FIG. 4 is displayed on a screen 14a of an LCD 14. As in the first embodiment, in the present embodiment, also, if only one comparison image is selected in a selection section 42a, the comparison image alone is displayed in an image display area 51C. If a plurality of comparison images are selected in the selection section 42a, one comparison image selected according to the degree of coincidence, from among the comparison images selected according to a selection condition, is displayed in the image display area 51C. In such a case, a user can cause a comparison image determined as having a highest degree of coincidence next to the currently displayed comparison image or a comparison image determined as having a lowest degree of coincidence next to the currently displayed comparison image to be displayed in the image display area 51C, by operating a predetermined operation member, for example, a joystick 4b of an operation portion 4.

Note that when two or more image display areas 51C that each display a comparison image are provided, two or more comparison images each having a high degree of coincidence are displayed in the plurality of image display areas 51C.

As described above, the above embodiment enables provision of an endoscope apparatus and an endoscope image display control method enabling a comparison image to be displayed easily.

Upon the user executing a comparison function during use of the measurement function in an examination, first, one or two or more images are extracted as comparison image(s) from among past endoscope images based on the system configuration information of the endoscope apparatus 1, and subsequently, one or two or more images are extracted as comparison image(s), from among the extracted one or two or more images based on the system configuration information, and the comparison image(s) are thus automatically searched for and displayed.

Then, if a plurality of comparison images are found based on the measurement information, a comparison image selected according to a predetermined selection condition is displayed in the image display area 51C of the image G2. When a plurality of comparison images are extracted, if the user wishes to cause another comparison image not displayed in the image display area 51C to be displayed in the image display area 51C of the image G2, the user can cause a next comparison image to be displayed in the image display area 51C, by operating the joystick 4b of the operation portion 4.

Therefore, upon execution of the comparison function by the user, a related past comparison image can be displayed in the image G2, which is a comparison function image, eliminating the need for complicated operation.

Next, modifications of the three embodiments described above will be described.

### (Modification 1)

Although the comparison function in each of the three embodiments described above causes a comparison image to be displayed side by side with a live image, for example, as disclosed in Japanese Patent Application Laid-Open Publication No. 2015-25965, a comparison image having a transparency that is not zero may be displayed so as to be superimposed on a live image. Even though a lightly transparent comparison image is superimposed on the live image, a user can determine a state of an examined site.

### (Modification 2)

The processing in the comparison image display control section 42, 42A, 42B may be fully or partly executed in a server connected to the endoscope apparatus 1 via a network.

The above is suitable for, for example, a case where past endoscope images are stored in the server connected via the network.

In other words, as indicated by dotted lines in FIG. 2, a body portion 3 includes a communication interface (I/F) 101. The communication interface 101 is communicably connected to a server 103 via a network 102 such as the Internet. The server 103 includes a storage apparatus 104, and a storage area 104a for programs for all or part of processing in the comparison image display control section 42, 42A, 42B is set in the storage apparatus 104. In other words, an endoscope system includes an endoscope apparatus 1 including an insertion portion 2 and the body portion 3, and the server 103 connected to the endoscope apparatus 1 via the network 102. The programs for all or part of the processing in the comparison image display control section 42, 42A, 42B are stored in the storage area 104a.

In the case of modification 2, the comparison image display control section 31d of a control section 31 provides an instruction to the server 103 to execute the programs for all or part of the processing in the comparison image display control section 42, 42A, 42B, the programs being stored in the storage area 104a.

More specifically, the comparison image display control section 31d includes a command signal transmission section 44 (indicated by dotted lines in FIG. 2) configured to transmit a predetermined command signal for providing an instruction to execute an extraction section 105, to the server 103 including the extraction section 105 (indicated by dotted lines in FIG. 2).

The server 103 includes the extraction section 105 configured to, upon receipt of a command signal from the endoscope apparatus 1, extract one or two or more images as one or two or more comparison images, from among a plurality of recorded images recorded in the storage apparatus 104, based on configuration information relating to a configuration of the endoscope apparatus 1 and/or measurement information relating to measurement processing on an endoscope image acquired in a CCD 21, and transmit the one or two or more comparison images.

The extracted one or two or more comparison images are transmitted to the endoscope apparatus 1 via the network 102. The comparison image display control section 42, 42A, 42B of the endoscope apparatus 1 causes an image including the one or two or more comparison images from the server 103 and the endoscope image obtained in the CCD 21 to be displayed on the LCD 14.

Modification 2 can also provide effects that are similar to the above-described effects of the three embodiments.

### (Modification 3)

Although in the three embodiments described above, system configuration information or measurement information are included in image data, it is possible that system configuration information, etc., is stored in association with image data, in, e.g., a table that is different from an image data file.

As above, above-described each of the embodiments and each of the modifications enable provision of an endoscope apparatus and an endoscope image display control method enabling a comparison image to be displayed easily.

The present invention is not limited to the above-described embodiments, and various changes, alterations and the like are possible without departing from the scope of the present invention.

What is claimed is:

1. An endoscope apparatus including an insertion portion and a body portion, the endoscope apparatus comprising:
an image sensor configured to pick up an image of a subject/object;
one or more controllers configured to:
extract one or two or more images as one or two or more comparison images, from among a plurality of recorded images recorded in a storage apparatus, based on first configuration information that is information relating to at least one of the insertion portion and the body portion and is configuration information indicating a configuration of the endoscope apparatus or first measurement information that is measurement information relating to measurement processing on an endoscope image acquired in the image sensor; and
perform display control to display an image including the one or two or more comparison images and the endoscope image on a display.

2. The endoscope apparatus according to claim 1, wherein the one or more controllers are configured to extract the one or two or more comparison images based on a degree of coincidence between the first configuration information and second configuration information that is the relevant configuration information added to each recorded image or a degree of coincidence between the first measurement information and second measurement information that is the relevant measurement information added to each recorded image.

3. The endoscope apparatus according to claim 1, wherein the one or more controllers are configured to record, in the storage apparatus, the acquired endoscope image with the first configuration information when the endoscope image is acquired added to the endoscope image as the second configuration information, or with the first measurement information relating to the measurement processing on the endoscope image added to the endoscope image as the second measurement information.

4. The endoscope apparatus according to claim 1, wherein each of the first configuration information and the second configuration information further includes information relating to an optical adapter fitted to a distal end portion of the insertion portion.

5. The endoscope apparatus according to claim 1, wherein each of the first measurement information and the second measurement information includes information relating to at least one of a measurement method and a measurement site designated in the measurement processing.

6. The endoscope apparatus according to claim 5, wherein each of the first measurement information and the second measurement information further includes information relating to a measurement result obtained in the measurement processing.

7. The endoscope apparatus according to claim 1, wherein after extraction of the one or two or more comparison images from among the plurality of recorded images based on the first configuration information, the one or more controllers are configured to extract the one or two or more comparison images from among the extracted one or two or more comparison images based on the first measurement information.

8. An endoscope system comprising an endoscope apparatus including an insertion portion and a body portion, and a server connected to the endoscope apparatus via a network, wherein:
the endoscope apparatus includes
an image sensor configured to pick up an image of a subject/object, and
one or more first controllers configured to transmit a predetermined command signal to the server;
the server includes
one or more second controllers configured to, upon reception of the command signal, extract one or two or more images as one or two or more comparison images, from among a plurality of recorded images recorded in a storage apparatus, based on first configuration information that is information relating to at least one of the insertion portion and the body portion and is configuration information indicating a configuration of the endoscope apparatus or first measurement information that is measurement information relating to measurement processing on an endoscope image acquired in the image sensor, and transmit the one or two or more images; and
the endoscope apparatus causes an image including the one or two or more comparison images from the server and the endoscope image to be displayed on a display of the endoscope apparatus.

9. An endoscope apparatus including an insertion portion and a body portion, the endoscope apparatus being connected to a server via a network, the endoscope apparatus comprising:
an image sensor configured to pick up an image of a subject/object; and
one or more controllers configured to transmit a command signal that provides an instruction to execute an extraction section configured to extract one or two or more images as one or two or more comparison images, from among a plurality of recorded images recorded in a storage apparatus, based on first configuration information that is information relating to at least one of the insertion portion and the body portion and is configuration information indicating a configuration of the endoscope apparatus or first measurement information that is measurement information relating to measurement processing on an endoscope image acquired by the image sensor, to the server including the extraction section,
wherein an image including the one or two or more comparison images extracted in the server and the endoscope image is displayed on a display of the endoscope apparatus.

10. An endoscope image display control method for an endoscope apparatus including an image sensor configured to pick up an image of a subject/object, an insertion portion and a body portion, the method comprising:
extracting one or two or more images as one or two or more comparison images, from among a plurality of recorded images recorded in a storage apparatus, based on first configuration information that is information relating to at least one of the insertion portion and the body portion and is configuration information indicating a configuration of the endoscope apparatus or first measurement information that is measurement information relating to measurement processing on an endoscope image acquired in the image sensor; and
displaying an image including the one or two or more comparison images and the endoscope image on a display.
